(19) United States
(12) Patent Application Publication
(10) Pub. No.: US 2020/0306290 A1
(43) Pub. Date: Oct. 1, 2020

(54) BEVERAGES FOR LOWERING BLOOD PRESSURE

(71) Applicant: 120 LIFE LLC, Chicago, IL (US)

(72) Inventors: Ira H. Antelis, Chicago, IL (US); Susan Schachter, Maplewood, NJ (US)

(21) Appl. No.: 16/830,904

(22) Filed: Mar. 26, 2020

Related U.S. Application Data
(60) Provisional application No. 62/825,987, filed on Mar. 29, 2019.

(51) Int. Cl. A61K 33/06; A61K 36/21; A61K 36/185; A61K 36/736; A61K 36/45; A61K 47/26; A61K 9/00; A61P 9/12

(52) U.S. Cl. CPC A61K 33/06; A61K 36/21; A61K 36/185; A61P 9/12; A61K 36/45; A61K 47/26; A61K 9/0095; A61K 36/736

(57) ABSTRACT

Embodiments of the present disclosure are directed to compositions, and in particular beverages, containing a blend of natural ingredients that, together, lower blood pressure when consumed regularly over a period of time. The natural ingredients may include beet root, pomegranate juice or puree, hibiscus, tart cherry juice or puree, and cranberry juice or puree. The beverage is formulated for taste and mouthfeel, as well as for effectiveness in lowering blood pressure. Embodiments of the present invention are also directed to methods of lowering blood pressure—systolic, diastolic, or both—by providing the beverage disclosed herein and instructing an individual to consume a serving of the beverage once per day for a period of time, for instance at least two weeks.

This application claims priority to U.S. Provisional Patent Application No. 62/825,987, filed Mar. 29, 2019, the entirety of which is incorporated by reference herein.

### BACKGROUND

According to recent statistics from the American Heart Association, over 100 million U.S. adults have high blood pressure, also known as hypertension. High blood pressure is generally defined as a systolic blood pressure of 130 or greater and/or a diastolic blood pressure of 80 or greater. High blood pressure is a common contributor to, or cause of, heart attacks, strokes, and other cardiovascular related events.

High blood pressure can be treated with medication. Medications for high blood pressure include diuretics, beta-blockers, ACE inhibitors, Angiotensin II receptor blockers, calcium channel blockers, alpha blockers, alpha-2 receptor agonists, central agonists, peripheral adrenergic inhibitors, vasodilators, and combinations of the above. Many of these medications have side effects, however. Moreover, many individuals forget to take their medications on a consistent basis, reducing their effectiveness, or simply stop taking their medications after a period of time (due for example to the side effects, forgetfulness, or any number of other reasons). Other individuals simply don't like taking medications or want to reduce the amount of foreign substances they are introducing into their bodies.

Many foods are known to have blood pressure lowering effects. However, it is difficult to manage one's blood pressure through the management of one's diet. Dietary supplements in pill form are also available, but like medication, individuals often forget to take pills. Many pill-based supplements also have ingredients that are not recognizable to a consumer and that may not come from natural sources. The effectiveness of some pill-based supplements has also been called into question.

### SUMMARY OF THE INVENTION

Embodiments of the present invention are directed to a nutritional composition, and in particular a beverage, that is configured to lower blood pressure. The beverage comprises a blend of two or more of the following natural ingredients: beet root powder, pomegranate juice or puree, hibiscus, tart cherry juice or puree, and cranberry juice of puree. The beverage may also comprise magnesium. The beverage may also include one or more sweeteners, one or more acid components such as citric acid to increase acidity, and one or more thickening components such as agar-agar to improve mouthfeel. The ingredients are provided in amounts such that, when consumed, the beverage both (a) is effective to lower blood pressure of a human subject and (b) has a desirable taste and mouthfeel. In some embodiments, the beverage may contain no artificial ingredients or synthetic active agents for lowering blood pressure.

Embodiments of the present invention are also directed to a method for lowering blood pressure in a human subject by providing the beverage of the present disclosure and instructing the subject to consume a serving of the beverage once per day for period of at least two weeks. The beverage may be provided in a plurality of containers, e.g. bottles (or packets if provided as a powdered beverage mix), each of which contains a daily serving. The amount of beverage that makes up a daily serving may vary depending on the degree of dilution of the active ingredients. For instance, a daily serving of the beverage may be between 2 fluid ounces and 16 fluid ounces, more desirably between 2 fluid ounces and 12 fluid ounces, more desirably between 2 fluid ounces and 10 fluid ounces.

### DETAILED DESCRIPTION OF THE INVENTION

Embodiments of the present disclosure are directed to nutritional compositions, and in particular beverages, for lowering blood pressure. In contrast to conventional methods of lowering blood pressure, the nutritional composition does not contain any pharmaceuticals, drugs, or other synthetic active agents. Instead, the composition consists solely of natural ingredients. It has presently been found that a combination of natural ingredients, when consumed together such as in a single beverage, may provide a synergistic and unexpected ability to lower blood pressure in a human. By consuming the beverage over a period of time, an individual may be able to significantly lower his or her blood pressure using natural-occurring ingredients and without ingesting drugs or synthetic compounds that may have harmful side effects.

Specifically, the beverages described herein have been found to have significant effects in lowering both an individual's systolic blood pressure, i.e. the first number of a conventional blood pressure measurement, and the individual's diastolic blood pressure, i.e. the second number of a conventional blood pressure measurement. For reference, systolic blood pressure (the first number) indicates how much pressure one's blood is exerting against one's artery walls when the heart beats. Diastolic blood pressure (the second number), on the other hand, indicates how much pressure one's blood is exerting against one's artery walls while the heart is resting between beats.

In addition to being formulated to provide significant blood pressure lowering effects described herein, the beverages described herein have also been formulated in order to have a good acidity and mouthfeel (e.g. viscosity), provide a good taste profile, and be transported and stored in large format and/or single-serve containers, e.g. bottles. By providing blood pressure lowering effects in a beverage that an individual will want to consume and that can be easily incorporated into one's daily diet, it is believed that individuals are more likely to consume the beverages consistently and over a longer period of time than might be the case with pills. It is also contemplated that individuals taking blood pressure medication may consume the beverage as a supplement along with (perhaps even at the same time as) their medication. The beverage may also be blended with other foods and/or juices, such as to create a smoothie or other blended beverage that will be enjoyable to consume.

The composition/beverage may include any combination of the following natural ingredients: beet root, pomegranate juice, hibiscus flower, tart (sour) cherry juice, and cranberry juice.

Beet root may be provided in any suitable form and may typically be provided as a water-soluble beet root powder. The use of other beet root sources or products, such as beet root purees, extracts, concentrates, or the like, is also contemplated. The beverage may contain beet root in any amount that is suitable for lowering a consumer's blood pressure. In some embodiments, the beverage may contain at least 0.5 tsp. beet root powder per daily serving (for example 8 fluid ounces); alternatively the beverage may comprise at least 1.0 tsp. beet root per daily serving; alternatively the beverage may comprise at least 1.5 tsp. beet root powder per daily serving; alternatively the beverage may comprise at least 2.0 tsp. beet root powder per daily serving. In some embodiments, the beverage may contain less than 5 tsp. beet root powder per daily serving (for example 8 fluid ounces), alternatively less than 4 tsp. beet root powder per daily serving, alternatively less than 3 tsp. beet root powder per daily serving.

The beet root powder is solubilized by the water and/or other liquid ingredients in the beverage, e.g. juices, to form a solution, e.g. an aqueous solution. This may be sufficient to provide at least 3,000 mg beet root per daily serving, alternatively at least 5,000 mg beet root per daily serving, alternatively at least 8,000 mg beet root per daily serving, alternatively at least 10,000 mg beet root per daily serving, alternatively at least 12,000 mg beet root per daily serving, alternatively at least 15,000 mg beet root per daily serving.

Pomegranate juice may be provided in any suitable form and may typically be provided as pomegranate juice concentrate. The use of other pomegranate juice sources or products, such as pomegranate purees, extracts, powders, or the like, is also contemplated. The beverage may contain pomegranate juice in any amount that is suitable for lowering a consumer's blood pressure. In some embodiments, the beverage may comprise at least 0.5 ounces of pomegranate juice or pomegranate juice concentrate per daily serving (for example 8 fluid ounces); alternatively the beverage may comprise at least 1.0 ounces of pomegranate juice or pomegranate juice concentrate per daily serving; alternatively the beverage may comprise at least 1.5 ounces of pomegranate juice or pomegranate juice concentrate per daily serving; alternatively the beverage may comprise at least 2.0 ounces of pomegranate juice or pomegranate juice concentrate per daily serving; alternatively the beverage may comprise at least 2.5 ounces of pomegranate juice or pomegranate juice concentrate per daily serving. In some embodiments, the beverage may contain less than 5 ounces of pomegranate juice or pomegranate juice concentrate per daily serving (for example 8 fluid ounces), alternatively less than 4 ounces, alternatively less than 3 ounces. This may be sufficient to provide at least 20,000 mg pomegranate per daily serving, alternatively at least 30,000 mg pomegranate per daily serving, alternatively at least 40,000 mg pomegranate per daily serving, alternatively at least 50,000 mg pomegranate per daily serving, alternatively at least 60,000 mg pomegranate per daily serving, alternatively at least 75,000 mg pomegranate per daily serving.

Hibiscus may be provided in any suitable form and may typically be provided as a water-soluble hibiscus flower powder or tea. The use of other hibiscus sources or products, such as hibiscus flower purees, extracts, concentrates, or the like, is also contemplated. The beverage may contain hibiscus powder or tea in any amount that is suitable for lowering a consumer's blood pressure. In some embodiments, the beverage may comprise at least 0.5 tsp. hibiscus powder per daily serving (for example 8 fluid ounces); alternatively the beverage may comprise at least 0.75 tsp. hibiscus powder per daily serving; alternatively the beverage may comprise at least 1 tsp. hibiscus powder per daily serving; alternatively the beverage may comprise at least 1.5 tsp. hibiscus powder per daily serving. In some embodiments, the beverage may contain less than 4 tsp hibiscus powder per daily serving (for example 8 fluid ounces), alternatively less than 3 tsp, alternatively less than 2 tsp.

The hibiscus flower powder or tea is solubilized by the water and/or other liquid ingredients in the beverage, e.g. juices, to form a solution, e.g. an aqueous solution. This may be sufficient to provide at least 1,000 mg hibiscus per daily serving, alternatively at least 2,000 mg hibiscus per daily serving, alternatively at least 3,000 mg hibiscus per daily serving, alternatively at least 4,000 mg hibiscus per daily serving, alternatively at least 5,000 mg hibiscus per daily serving, alternatively at least 6,000 mg hibiscus per daily serving.

Tart (or sour) cherry juice may be provided in any suitable form and may typically be provided as tart cherry juice concentrate. The use of other tart cherry juice sources or products, such as tart cherry purees, extracts, powders, or the like, is also contemplated. The beverage may contain tart cherry juice in any amount that is suitable for lowering a consumer's blood pressure. In some embodiments, the beverage may comprise at least 0.25 ounces of tart cherry juice or tart cherry juice concentrate per daily serving (for example 8 fluid ounces); alternatively the beverage may comprise at least 0.5 ounces of tart cherry juice or tart cherry juice concentrate per daily serving; alternatively the beverage may comprise at least 0.75 ounces of tart cherry juice or tart cherry juice concentrate per daily serving; alternatively the beverage may comprise at least 1.0 ounces of tart cherry juice or tart cherry juice concentrate per daily serving; alternatively the beverage may comprise at least 1.25 ounces of tart cherry juice or tart cherry juice concentrate per daily serving. In some embodiments, the beverage may contain less than 5 ounces of tart cherry juice or tart cherry juice concentrate per daily serving (for example 8 fluid ounces), alternatively less than 4 ounces, alternatively less than 3 ounces. This may be sufficient to provide at least 10,000 mg tart cherry per 8 ounces of beverage, alternatively at least 15,000 mg tart cherry per daily serving, alternatively at least 20,000 mg tart cherry per daily serving, alternatively at least 25,000 mg tart cherry per daily serving, alternatively at least 30,000 mg tart cherry per daily serving, alternatively at least 35,000 mg tart cherry per daily serving.

Cranberry juice may be provided in any suitable form and may typically be provided as cranberry juice concentrate. The use of other cranberry juice sources or products, such as cranberry purees, extracts, powders, or the like, is also contemplated. The beverage may contain cranberry juice in any amount that is suitable for lowering a consumer's blood pressure. In some embodiments, the beverage may comprise at least 0.25 ounces of cranberry juice or cranberry juice concentrate per daily serving (for example 8 fluid ounces); alternatively the beverage may comprise at least 0.5 ounces of cranberry juice or cranberry juice concentrate per daily serving; alternatively the beverage may comprise at least 0.75 ounces of cranberry juice or cranberry juice concentrate per daily serving; alternatively the beverage may comprise at least 1.0 ounces of cranberry juice or cranberry juice concentrate per daily serving; alternatively the beverage may comprise at least 1.125 ounces of cranberry juice or cranberry juice concentrate per daily serving. In some embodiments, the beverage may contain less than 5 ounces of cranberry juice or cranberry juice concentrate per daily serving (for example 8 fluid ounces), alternatively less than 4 ounces, alternatively less than 3 ounces. This may be sufficient to provide at least 2,000 mg cranberry per daily serving, alternatively at least 3,000 mg cranberry per daily serving, alternatively at least 4,000 mg cranberry per daily serving, alternatively at least 5,000 mg cranberry per daily serving, alternatively at least 6,000 mg cranberry per daily serving, alternatively at least 7,000 mg cranberry per daily serving.

In some embodiments, the composition/beverage may also include magnesium. For instance, the beverage may contain up to 100% of a person's daily recommended dosage of magnesium per daily serving (for example 8 fluid ounces). For instance, the beverage may contain at least 100 mg magnesium per daily serving; alternatively the beverage may contain at least 200 mg magnesium per daily serving; alternatively the beverage may contain at least 300 mg magnesium per daily serving. In some embodiments, the beverage may contain less than 500 mg magnesium per daily serving, alternatively less than 400 mg per daily serving. The magnesium may be provided in any suitable nutritional form.

In some embodiments, the composition/beverage may also include a small amount of sweetener, such as a natural sugar sweetener or a sugar substitute sweetener, to provide the beverage with a desired taste. In some embodiments, for instance, the composition/beverage may include a small amount of a stevia sweetener. The stevia sweetener may be provided in any suitable form, such as stevia herbal extract powder. In some embodiments, the beverage may contain between 0.05 and 0.2 tsp stevia powder, alternatively between 0.05 and 0.15 tsp stevia powder, alternatively between 0.075 and 0.2 tsp stevia powder, alternatively between 0.075 and 0.15 tsp stevia powder, alternatively between 0.1 tsp and 0.2 tsp stevia powder, alternatively between 0.1 and 0.15 tsp stevia powder. This may be sufficient to provide between about 0.5 mg and about 5 mg stevia, alternatively between about 1 mg stevia and about 4 mg stevia, alternatively between about 2 mg stevia and about 3 mg stevia.

In some embodiments, the composition/beverage may also include a small amount of an ingredient designed to increase the acidity of the beverage in order to provide the beverage with a desirable taste. In some embodiments, for instance, the composition/beverage may include a small amount of an acidifying agent such as citric acid.

In some embodiments, the composition/beverage may also include a small amount of an ingredient designed to improve the mouthfeel, or texture, of the beverage. In some embodiments, for instance, the composition/beverage may include a small amount of a thickening agent, such as agar agar.

The above-ingredients may be mixed together with water or carbonated water (seltzer water) to produce a beverage in a conventional manner. The beverage may be formulated (e.g. by the addition of water or carbonated water) to provide a daily serving in a suitable and desirable volume for single-serve consumption. For example, the daily serving may be 2 fluid ounces, 2.5 fluid ounces, 3 fluid ounces, 4 fluid ounces, 5 fluid ounces, 6 fluid ounces, 7 fluid ounces, 8 fluid ounces, 9 fluid ounces, 10 fluid ounces, 11 fluid ounces, 12 fluid ounces, or the like. In some embodiments, the beverage may be formulated so that the daily serving is 8 fluid ounces, provided in a single-serve 8 ounce bottle. In other embodiments, the beverage may be formulated so that the daily serving is 2.5 fluid ounces, provided in a single-serve 2.5 ounce bottle.

In some embodiments, the beverage may contain less than 120 calories per daily serving, alternatively less than 110 calories per daily serving, alternatively less than 100 calories per daily serving, alternatively less than 95 calories per daily serving, alternatively less than 90 calories per daily serving. In some embodiments, the beverage may contain zero fat.

The beverage may be placed into bottles or other containers of any conventional size. In some embodiments, a recommended daily serving of the beverage may be made available in a single-serving bottle (or other container). For example, the beverage may be sold as 2.5 ounce bottles, 3 ounce bottles, 6 ounce bottles, as 8 ounce bottles, as 10 ounce bottles, as 12 ounce bottles, or the like. Individual serving bottles may, of course, be sold in multiple-bottle packs, e.g. four packs, six packs, seven (week) packs, and the like. To reduce packaging materials and product waste, the beverage may also be made available in larger containers that include multiple daily servings, such as 16 ounce bottles, 24 ounce bottles, 32 ounce bottles, 40 ounce bottles, gallon bottles/jugs, and the like.

It is also contemplated that in some embodiments the beverage may be spray-dried to provide a powder, which may then be reconstituted with water by a user. The powdered product may be sold in single-use disposable packets and/or in larger-format containers/cans, from which a user may measure a daily recommended amount for reconstitution with water.

The beverage may be configured for a user to consume a serving size once per day, with the daily serving size being suitable to provide the blood pressure lowering effect. An individual may therefore be instructed to consume a serving size, such as a single-serving bottle or a measured amount from a bulk-serving bottle, once per day. In other embodiments, an individual may be instructed to consume multiple smaller servings each day, a larger serving size every two days, or the like. When these instructions are followed so that an appropriate amount of beverage is consumed each day, such as in a nutritional regimen or dietary program, blood pressure lowering effects are generally achieved within a week or within two weeks. By drinking a daily serving size of the beverage every day over a period of time, i.e. weeks, months, or years, an individual may be able to reduce and then maintain his or her blood pressure at a healthy level—close to 120/80 (or less)—over that period of time.

### Example 1 (Sample Beverage Composition)

A sample beverage was prepared from the following ingredients: Water, Pomegranate Juice, Sour Cherry Juice, Cranberry Juice, Beet Root, Hibiscus Flower, Citric Acid, Magnesium Chelate, Stevia, and Agar Agar. The sample beverage was bottled in 8 oz. bottles, such that a daily serving size of 8 ounces could be easily consumed in one sitting.

The beverage included the following ingredients per 8 ounce serving, all of which were mixed together with water to prepare the beverage:
about 2 tsp. of beet root powder, obtained from Quick Dry Foods (about 15,000 mg);
about 2.5 ounces of pomegranate juice concentrate, obtained from Greenwood Associates (about 77,500 mg);
about 1.5 tsp. of organic hibiscus flower powder, obtained from StarWest Botanicals (about 6,150 mg);
about 1.25 ounces of tart (sour) cherry juice concentrate, obtained from Greenwood Associates (about 35,000 mg);
about 1.125 ounces of cranberry juice concentrate, obtained from Greenwood Associates (about 7,031 mg);
about 1.5 tsp. of magnesium chelate 20%, obtained from Nutrisol Solutions (about 4,987 mg);
about 0.125 tsp. of stevia herbal extract powder, obtained from Zhucheng Haotian Pharm Co. Ltd, for sweetness (about 2.5 mg);
small amounts of citric acid for acidity and agar agar for mouthfeel

| | Percent of |
|---|---|
| Ingredient | Formula |
| Water | 85.4521 |
| Pomegranate Juice Concentrate | 5.7134 |
| Tart Cherry Juice Concentrate | 2.8567 |
| Cranberry Juice Concentrate | 2.4210 |
| Organic Hibiscus Flower Powder | 1.0775 |
| Beet Root Powder | 1.1360 |
| Magnesium Chelate | 0.5550 |
| Stevia | 0.0600 |
| Citric Acid | 0.7083 |
| Agar Agar | 0.0200 |

The beverage from this example was packaged into 8 fluid ounce bottles.

### Example 2 (sample Beverage Composition)

A more concentrated sample beverage was prepared from the following ingredients: Pomegranate Juice, Sour Cherry Juice, Cranberry Juice, Beet Root, Hibiscus Flower, Citric Acid, Magnesium Chelate, and Stevia. The concentrated sample beverage was bottled in 2.5 fluid ounce bottles, such that the same daily serving of the ingredients provided in the 8 fluid ounce bottle of Example 1 could be easily consumed on the go.

The beverage included the following ingredients, all of which were mixed together with water to prepare the beverage:
about 2 tsp. of beet root powder, obtained from Quick Dry Foods (about 15,000 mg);
about 2.5 ounces of pomegranate juice concentrate, obtained from Greenwood Associates (about 77,500 mg);
about 1.5 tsp. of organic hibiscus flower powder, obtained from StarWest Botanicals (about 6,150 mg);
about 1.25 ounces of tart (sour) cherry juice concentrate, obtained from Greenwood Associates (about 35,000 mg);
about 1.125 ounces of cranberry juice concentrate, obtained from Greenwood Associates (about 7,031 mg);
about 1.5 tsp. of magnesium chelate 20%, obtained from Nutrisol Solutions (about 4,987 mg);
about 0.125 tsp. of stevia herbal extract powder, obtained from Zhucheng Haotian Pharm Co. Ltd, for sweetness (about 2.5 mg);
small amounts of citric acid for acidity
   The beverage from this example was packaged into 2.5 fluid ounce bottles.

### Example 3 (Test Results)

A number of individuals, each of who had elevated or high blood pressure, were instructed to consume one eight-ounce bottle of the beverage from Example 1 per day for a period of time of at least two weeks. Each individual's blood pressure was measured prior to the initiation of the study and is listed below in the column titled “BP Before”. Each individual's blood pressure was again measured at some point during or after the two weeks and the results are listed below in the column titled “BP After”. As can be seen from the below test results, the beverage from Example 1 was shown to have a significant blood pressure lowering effect.

| | | | | % | % |
|---|---|---|---|---|---|
| | | | | reduction | reduction |
| Individual | BP Before | BP After | Timeframe | systolic | diastolic |
| Individual 1 | 155/91 | 132/80 | within a | 14% | 12% |
| | | | month | | |
| Individual 2 | 130+/80+ | 120/80 | four days | 7% | 0% |
| Individual 3 | 200/100 | 118/79 | three weeks | 41% | 21% |
| Individual 4 | 134/81 | 119/68 | a week | 11% | 16% |
| Individual 5 | 128/79 | 116/71 | 8 days | 9% | 10% |
| Individual 6 | 194/114 | 134/83 | one week | 30% | 27% |
| Individual 7 | 145/89 | 111/76 | about one | 23% | 14% |
| | | | month | | |
| Individual 8 | 147/90 | 126/82 | | 14% | 8% |
| Individual 9 | 198/127 | 116/65 | | 41% | 48% |
| Individual 10 | 180/95 | 135/93 | | 25% | 2% |
| Individual 11 | 160/101 | under | | 25% | 20% |
| | | 120/80 | | | |
| Individual 12 | 149/85 | 128/80 | | 14% | 5% |
| Individual 13 | systolic | systolic | week and | 28% | |
| | 170 | 122 | a half | | |
| Individual 14 | systolic | 120/80 | | 14% | |
| | 140-160 | | | | |
| Individual 15 | systolic | 128/74 | | 31% | |
| | 188-190 | | | | |
| Individual 16 | systolic | systolic | | 10% | |
| | 140+ | 110-125 | | | |

While there is a relatively large variance in the exact effects between different individuals, the above test data demonstrates a consistent and notable reduction in both systolic and diastolic blood pressure measurements. In some embodiments, for example, the beverages of the present disclosure may provide at least a 5% reduction in systolic blood pressure when consumed at least once per day for a period of time, such as two weeks, alternatively at least a 10% reduction in systolic blood pressure, alternatively at least a 15% reduction in systolic blood pressure, alternatively at least a 20% reduction in systolic blood pressure, alternatively at least a 25% reduction in systolic blood pressure. In some embodiments, for example, the beverages of the present disclosure may provide at least a 5% reduction in diastolic blood pressure when consumed once per day for a period of time, such as two weeks, alternatively at least a 10% reduction in diastolic blood pressure, alternatively at least a 15% reduction in diastolic blood pressure, alternatively at least a 20% reduction in diastolic blood pressure, alternatively at least a 25% reduction in diastolic blood pressure.

In some embodiments, the beverages of the present disclosure, when consumed as least once per day over a period of time, may be effective to maintain an individual's blood pressure within a healthy range, such as 120/80 or below. In some embodiments for example, the beverages of the present disclosure may be effective to reduce an individual's systolic blood pressure from a value above 120 to 120 or below, e.g. from at least 130 to 120 or below, from at least 135 to 120 or below, from at least 140 to 120 or below, from at least 145 to 120 or below, from at least 150 to 120 or below, etc. In some embodiments, the beverages of the present disclosure may also be effective to reduce an individual's diastolic blood pressure from a value above 80 to 80 or below, e.g. from at least 85 to 80 or below, from at least 90 to 80 or below, from at least 95 to 80 or below, from at least 100 to 80 or below, etc. By continuing to consume the beverage at least once a day, the lowered blood pressure may be maintained over an extended period of time, such as over a number of years.

Embodiments of the present disclosure are also directed to non-therapeutic methods for lowering blood pressure in a human subject, such as an individual that suffers from high blood pressure. The method comprises providing the composition, and in particular the beverage, disclosed herein, and instructing the subject to consume a daily serving of the composition/beverage each day for a period of time of at least two weeks. In some embodiments, the daily serving may be provided by a single-serving beverage. The daily serving may be formulated as an amount of beverage that may easily be consumed at one time, for instance, between 1 fluid ounce and 16 fluid ounces, between 2 fluid ounces and 12 fluid ounces, between 2 fluid ounces and 10 fluid ounces, etc. In some embodiments the beverage may be provided in a plurality of containers, e.g. bottles, with each container providing the daily serving.

After consuming the beverage in accordance with the instructions and method described above, the subject's systolic blood pressure reading may be reduced by at least 5%, alternatively at least 10%, alternatively at least 15%, alternatively at least 20%, alternatively at least 25%. In some embodiments, a subject's systolic blood pressure may be greater than 120 prior to consuming the beverage in accordance with the instructions and method described above (e.g. greater than 130, greater than 135, greater than 140, greater than 145, greater than 150), and after consuming the beverage in accordance with the instructions and method described above, the subject's systolic blood pressure may be 120 or less. By a continued consumption of the beverage in accordance with the method and instructions described above, the subject's systolic blood pressure may be maintained at 120 or less.

After consuming the beverage in accordance with the instructions and method described above, the subject's diastolic blood pressure reading may be reduced by at least 5%, alternatively at least 10%, alternatively at least 15%, alternatively at least 20%, alternatively at least 25%. In some embodiments, a subject's diastolic blood pressure may be greater than 80 prior to consuming the beverage in accordance with the instructions and method described above (e.g. greater than 85, greater than 90, greater than 95, greater than 100), and after consuming the beverage in accordance with the instructions and method described above, the subject's diastolic blood pressure may be 80 or less. By a continued consumption of the beverage in accordance with the method and instructions described above, the subject's diastolic blood pressure may be maintained at 80 or less.

It can be seen that the described embodiments provide unique and novel blood-pressure lowering beverages that have a number of advantages over other treatments in the art. While there is shown and described herein certain specific embodiments embodying the invention, it will be manifest to those skilled in the art that various modifications may be made without departing from the spirit and scope of the underlying inventive concept and that the same is not limited to the particular forms herein shown and described except insofar as indicated by the scope of the appended claims.

What is claimed:

1. A beverage configured to lower blood pressure in a human, comprising:
a. beet root;
b. pomegranate juice or puree; and
c. hibiscus.

2. The beverage of claim 1, further comprising tart cherry juice or puree.

3. The beverage of claim 2, further comprising cranberry juice or puree.

4. The beverage of claim 3, further comprising magnesium.

5. The beverage of claim 4, further comprising stevia.

6. The beverage of claim 1, wherein the beet root is present in an amount of at least 5,000 mg per serving.

7. The beverage of any one of claim 6, wherein the beet root is water-soluble beet root powder.

8. The beverage of claim 1, wherein the pomegranate juice or puree is present in an amount of at least 1.5 ounces per serving.

9. The beverage of claim 8, wherein the pomegranate juice or puree is pomegranate juice concentrate or extract.

10. The beverage of claim 1, wherein the hibiscus is present in an amount of at least 3,000 mg per serving.

11. The beverage of claim 10, wherein the hibiscus is water-soluble hibiscus powder.

12. The beverage of claim 2, wherein the tart cherry juice or puree is present in an amount of at least 0.75 ounces per serving.

13. The beverage of claim 12, wherein the tart cherry juice or puree is tart cherry juice concentrate or extract.

14. The beverage of claim 3, wherein the cranberry juice or puree is present in an amount of at least 0.75 ounces per serving.

15. The beverage of claim 14, wherein the cranberry juice or puree is cranberry juice concentrate or extract.

16. The beverage of claim 4, wherein the beverage contains at least 200 mg magnesium per serving.

17. The beverage of claim 5, wherein the beverage contains at least 1.0 mg stevia per serving.

18. The beverage of claim 1, further comprising one or more thickening agents.

19. The beverage of claim 1, further comprising one or more acidifying agents.

20. The beverage of claim 5, wherein the beverage contains no artificial ingredients for lowering blood pressure.

21. The beverage of claim 1, wherein a serving of the beverage is between 2 fluid ounces and 12 fluid ounces.

22. A method for lowering blood pressure in a human subject comprising:
a. providing the beverage of claim 21; and
b. instructing the subject to consume a serving of the beverage once per day for a period of time of at least two weeks.

23. The method of claim 22, wherein the beverage is provided in a plurality of bottles, with each bottle containing a single serving.

24. The method of claim 22, wherein after consuming the beverage in accordance with the instructions of step b. for a period of two weeks, the subject's systolic blood pressure reading is reduced by at least 5%.

25. The method of claim 22, wherein after consuming the beverage in accordance with the instructions of step b. for a period of two weeks, the subject's diastolic blood pressure reading is reduced by at least 5%.
